(19) United States
(12) Patent Application Publication
(10) Pub. No.: US 2002/0049250 A1
(43) Pub. Date: Apr. 25, 2002

(54) PPAR receptor activator compounds for treating cutaneous disorders/afflictions

(76) Inventors: Jean Maignan, Tremblay-En-France (FR); Serge Michel, Roquefort-Les-Pins (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/933,835

(22) Filed: Aug. 22, 2001

Related U.S. Application Data
(63) Continuation of application No. PCT/FR00/03645, filed on Dec. 21, 2000.

(30) Foreign Application Priority Data
Dec. 22, 1999 (FR) 99/16269

(51) Int. Cl. A61K 31/38; A61K 31/385; A61K 31/195; A61K 31/192

(52) U.S. Cl. 514/434; 514/432; 514/456; 514/567; 514/569

(57) ABSTRACT

Compounds of formula (I):

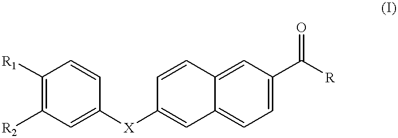

characteristically activators of receptors of PPAR type, are well suited for treating cutaneous disorders/afflictions, notably rosacea, intrinsic (or chronological) aging, pigmentation disorders, seborrhoeic function disorders, barrier function disorders and more particularly epidermal lipid secretion disorders, wound healing disorders, corticosteroid-induced cutaneous atrophies, or ulcers, immune system disorders, cardiovascular system conditions, and/or lipid metabolism disorders.

### CROSS-REFERENCE TO PRIORITY/PCT APPLICATIONS

This application claims priority under 35 U.S.C. §119 of FR-99/16269, filed Dec. 22, 1999, and is a continuation of PCT/FR00/03645, filed Dec. 21, 2000 and designating the United States (published in the French language on Jun. 28, 2001 as WO 01/45663 A2; the title and abstract were also published in English), both hereby expressly incorporated by reference.

### CROSS-REFERENCE TO COMPANION APPLICATION

Copending application Serial No. ______ [Attorney Docket No. 016800-451], filed concurrently herewith and assigned to the assignee hereof.

### BACKGROUND OF THE INVENTION

### TECHNICAL FIELD OF THE INVENTION

The present invention relates to the administration of polycyclic aromatic compounds or cosmetic/pharmaceutical compositions comprised thereof, for treating skin disorders/afflictions such as rosacea, intrinsic (or chronological) aging, pigmentation disorders, seborrhoeic function disorders, barrier function disorders and more particularly epidermal lipid secretion disorders, wound healing disorders, corticosteroid-induced cutaneous atrophies, or ulcers; immune system disorders; cardiovascular system conditions, and/or lipid metabolism disorders.

The present invention also relates to a cosmetic/pharmaceutical regime or regimen for restoring the barrier function of the skin and more particularly for regulating and/or restoring the metabolism of cutaneous lipids, comprising topically applying onto the skin at least one compound of formula (I) below, and more particularly as a PPAR-type receptor activator, or composition comprised thereof.

### SUMMARY OF THE INVENTION

It has now surprisingly and unexpectedly been determined that certain polycyclic aromatic compounds, notably those described in EP-220,118, U.S. Pat. No. 5,023,363 and FR-2,600,064 exhibit an antiproliferative action and elicit marked activity on the transactivation of PPAR-type receptors.

The present invention thus features administration of an effective amount of at least one polycyclic aromatic compound, and more particularly as PPAR-type receptor activators, or compositions comprised thereof, for treating skin disorders/afflictions such as rosacea, intrinsic (or chronological) aging, pigmentation disorders, seborrhoeic function disorders, barrier function disorders and more particularly epidermal lipid secretion disorders, wound healing disorders, corticosteroid-induced cutaneous atrophies, or ulcers; immune system disorders; cardiovascular system conditions, and/or lipid metabolism disorders.

This invention also features a cosmetic regime/regimen for restoring the barrier function of the skin and more particularly for regulating and/or restoring the metabolism of cutaneous lipids, comprising topically applying onto the skin at least one compound of formula (I), and more particularly as a PPAR-type receptor activator.

The subject compounds have the structural formula (I):

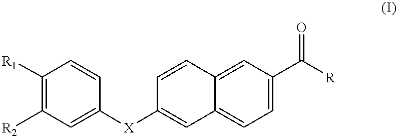

in which R is a hydrogen atom or an —OR₃ radical, wherein R₃ is as defined below; X is a ═C═O radical or a ═C═N—OH radical; R₁ and R₂, which may be identical or different, are each a hydrogen atom, a linear or branched alkyl radical having from 1 to 12 carbon atoms, an —OR₄ radical, an —S(O)ₙR₄ radical, an —OCOR₄ radical, or an —NHCOR₄ radical, n and R₄ are as defined below, with the proviso that R₁ and R₂ may together form with the adjacent aromatic ring a 5- or 6-membered saturated or unsaturated ring optionally substituted with methyl groups and/or optionally interrupted by an oxygen or sulfur atom, a sulfone radical or a sulfoxide radical, said ring preferably being saturated, and with the further proviso that, when X is a ═C ═O radical, then R₁ and R₂ do not form with the adjacent aromatic ring a 5-membered ring substituted with methyl groups; R₃ is a hydrogen atom, a linear or branched alkyl radical having from 1 to 20 carbon atoms, or a mono- or polyhydroxyalkyl radical; n is 0, 1 or 2; R₄ is a hydrogen atom, a lower alkyl radical, or a phenyl radical; and include the salts and/or chiral analogs thereof. Such salts include those of an alkali or alkaline earth metal, or of zinc, or of an organic amine.

### DETAILED DESCRIPTION OF BEST MODE AND SPECIFIC/PREFERRED EMBODIMENTS OF THE INVENTION

More particularly according to the present invention, by the expression “lower alkyl radical” is intended a radical having from 1 to 6 carbon atoms, preferably methyl, ethyl, isopropyl, butyl, tert-butyl and hexyl radicals.

And exemplary linear or branched alkyl radicals having from 1 to 12 carbon atoms are methyl, ethyl, propyl, 2-ethylhexyl, octyl and dodecyl radicals.

Among the linear or branched alkyl radicals having from 1 to 20 carbon atoms, exemplary are methyl, ethyl, propyl, 2-ethylhexyl, octyl, dodecyl, hexadecyl and octadecyl radicals.

By the expression “monohydroxyalkyl radical” is intended a radical having 1 to 6 carbon atoms and preferably having from 2 to 3 carbon atoms, in particular a 2-hydroxyethyl, 2-hydroxypropyl or 3-hydroxypropyl radical.

By the expression “polyhydroxyalkyl radical” is intended a radical having from 3 to 6 carbon atoms and from 2 to 5 hydroxyl groups, such as 2,3-dihydroxypropyl, 2,3,4-trihydroxybutyl or 2,3,4,5-tetrahydroxypentyl radicals, or the pentaerythritol residue.

Particularly exemplary compounds of formula (I) are the following:

Compound 1: 6-(5,6,7,8-tetrahydro-5,5,8,8-tetramethylnaphthalene-2-carbonyl)naphthalene-2-carboxylic acid;

Compound 2: 6-(2,2-dimethylchroman-6-carbonyl)naphthalene-2-carboxylic acid;

Compound 3: 6-(4-tert-butylbenzoyl)naphthalene-2-carboxylic acid;

Compound 4: 6-(5,6,7,8-tetrahydronaphthalene-2-carbonyl)naphthalene-2-carboxylic acid;

Compound 5: 6-(4,4-dimethylchroman-6-carbonyl)naphthalene-2-carboxylic acid; and

Compound 6: 6-[hydroxyimino(5,5,8,8-tetramethyl-5,6,7,8-tetrahydronaphthalen-2-yl)methyl]naphthalene-2-carboxylic acid.

According to the present invention, the compound of formula (I) which is more particularly preferred is Compound 3:

6-(4-tert-butylbenzoyl)naphthalene-2-carboxylic acid.

The compounds of formula (I) can be prepared, in particular, by the methods of preparation described in EP-220,118, U.S. Pat. No. 5,023,363 and FR-2,600,064.

The compounds of the invention exhibit PPAR-type receptor activation properties. The PPAR-type receptors are receptors which belong to the family of nuclear steroid receptors.

By the expression “PPAR-type receptor activator” according to the invention is intended any compound which exhibits, in a transactivation test, as dscribed in Kliewer et al., Nature, 358, 771-774 (1992), an AC₅₀ of less than or equal to 10 μm. Preferably, the PPAR-type receptor activator exhibits an AC₅₀ of less than or equal to 2 μm and advantageously of less than or equal to 1 μm.

An AC₅₀ is the concentration of “activator” compound necessary to exhibit 50% of the activity of a reference molecule. This activity is determined with the aid of a reporter enzyme (luciferase) for activation due to the compound via one of the PPAR receptors.

The activity of the PPAR-type receptors has been the subject of numerous studies and publications. Exemplary is the publication entitled “Differential Expression of Peroxisome Proliferator-Activated Receptor Subtypes During the Differentiation of Human Keratinocytes”, Michel Rivier et al., J. Invest. Dermatol., 111, p 1116-1121 (1998), in which a large number of bibliographical references relating to the PPAR-type receptors are listed.

The use of PPAR-α-type receptor activators for restoring the barrier function and more particularly epidermal lipid secretion disorders, for promoting differentiation and for inhibiting epidermal proliferation has been described in WO 98/32444.

Furthermore, the administration of PPAR-α- and/or PPAR-γ-type receptor activators for treating cutaneous disorders related to abnormality in the differentiation of epidermal cells has been described in the publication by Michel Rivier et al., J. Invest. Dermatol., 111, p 1116-1121 (1998).

It has also been described in WO 96/33724 that compounds selective for PPARγ-receptors, such as prostaglandin-J2 or -D2, are potential active agents for the treatment of obesity and diabetes.

Pharmaceutical compositions comprising at least one compound of formula (I) are thus well suited for the treatment of rosacea, pigmentation disorders, seborrhoeic function disorders, barrier function disorders and more particularly epidermal lipid secretion disorders, wound healing disorders, corticosteroid-induced cutaneous atrophies, or ulcers; immune system disorders; cardiovascular system conditions, and/or lipid metabolism disorders.

By the expression “pigmentation disorders” are intended, in particular, hyperpigmentation, melasma, hypopigmentation or vilitigo. Preferably, the pigmentation disorders are not associated with the harmful effects of the sun (the damaging effects of solar radiation).

Exemplary seborrhoeic function disorders are, in particular, hyperseborrhoeic or seborrhoeic dermatitis.

Particularly exemplary barrier function disorders of the skin and more particularly the epidermal lipid secretion disorders are skin disorders in premature children born before 33 weeks, lip fissures, chapped lips or blisters resulting from mechanical friction.

Exemplary wound healing disorders are, in particular, keloids or hypertrophic scars.

Exemplary ulcers are, ulcers and erosions due to chemical or heat burns, bullous disorders, or vascular disorders or ischaemia, including venous, arterial, embolic or diabetic ulcers.

Exemplary immune system disorders are, in particular, autoimmune diseases (such as, but without limitation, type 1 diabetes mellitus, multiple sclerosis, lupus and lupus-type conditions, glomerulonephritis, and the like), or selective immune system dysfunctions (for example AIDS).

Exemplary cardiovascular system conditions include atherosclerosis or hypertension.

And exemplary lipid metabolism conditions include obesity, hyperlipidaemia or non-insulin-dependent diabetes.

The compositions according to the invention can be administered via the enteral, parenteral or topical or ocular route, for such period of time as required to elicit the desired response. The pharmaceutical compositions are preferably packaged in a form suitable for application by the topical route.

The subject compositions, more particularly the pharmaceutical compositions, can be provided, for the enteral route, in the form of tablets, including sugar-coated tablets, hard gelatin capsules, syrups, suspensions, solutions, powders, granules, emulsions or lipid or polymeric microspheres or nanospheres or vesicles which permit controlled release. The subject compositions can be provided, for the parenteral route, in the form of solutions or suspensions for infusion or for injection.

The subject compounds according to the invention are generally administered at a daily dose of approximately 0.001 mg/kg to 100 mg/kg of body weight, taken on 1 to 3 occasions.

The pharmaceutical compositions according to the invention, for the topical route, are more preferably for the treatment of the skin and mucous membranes and can be provided in the form of salves, creams, emulsions, milks, ointments, powders, impregnated pads, solutions, gels, sprays, lotions or suspensions. Same can also be provided in the form of lipid or polymeric microspheres or nanospheres or vesicles or of polymeric patches and hydrogels which permit controlled release. The compositions for topical application can be provided either in anhydrous form or in aqueous form.

The subject compounds are administered via the topical route at a concentration generally ranging from 0.001% to 10% by weight, preferably from 0.01 to 1% by weight, with respect to the total weight of the composition.

The compounds of formula (I) according to the invention also find application in the cosmetics field, in particular in body and hair hygiene and more particularly for restoring the barrier function of the skin and more particularly for regulating and/or restoring the metabolism of cutaneous lipids. In comparison with the products known previously, these compounds of formula (I) present the advantage of additionally exhibiting other advantageous properties, in particular anti-inflammatory or soothing properties, which makes them less irritating and therefore better tolerated compounds.

The cosmetic compositions according to the invention, comprising, in a cosmetically acceptable vehicle, diluent or carrier, at least one compound of formula (I), one of its optical or geometrical isomers or one of its salts, can be provided, in particular, in the form of a cream, milk, lotion, emulsion, gel, lipid or polymeric microspheres or nanospheres or vesicles, soap or shampoo.

The concentration of compound of formula (I) in the cosmetic compositions advantageously ranges from 0.001% to 3% by weight.

The compositions according to the present invention can, of course, additionally comprise inert or even pharmacodynamically active additives or adjuvants, or combinations of these additives and adjuvants, and in particular: wetting agents; depigmenting agents, such as hydroquinone, azelaic acid, caffeic acid or kojic acid; emollients; moisturizing agents, such as glycerol, PEG 400, thiamorpholinone and derivatives thereof, or urea; antiseborrhoeic or antiacne agents, such as S-carboxymethylcysteine, S-benzylcysteamine, their salts or derivatives thereof, or benzoyl peroxide; antifungal agents, such as ketoconazole or 4,5-polymethylene-3-isothiazolidones; antibacterials; carotenoids and in particular β-carotene; antipsoriatic agents, such as anthralin and derivatives thereof; eicosa-5,8,11,14-tetraynoic and eicosa-5,8,11-triynoic acids, their esters and amides, and also the retinoids. The subject compounds of formula (I) can also be formulated with vitamins D or derivatives thereof, with corticosteroids, with agents for combating free radicals, with α-hydroxy or α-keto acids or derivatives thereof, or with ion channel blockers.

These compositions can also comprise flavor-improving agents, preservatives, such as esters of para-hydroxybenzoic acid, stabilizing agents, moisture-regulating agents, pH-regulating agents, agents for modifying osmotic pressure, emulsifying agents, UV-A and UV-B screening agents, or antioxidants, such as α-tocopherol, butylhydroxyanisole or butylhydroxy-toluene.

One skilled in this art will of course take care to select the optional compound or compounds to be added to these compositions such that the advantageous properties intrinsically associated with the present invention are not, or not substantially, detrimentally affected by the envisaged addition.

In order to further illustrate the present invention and the advantages thereof, the following specific examples are given, it being understood that same are intended only as illustrative and in nowise limitative.

In said examples to follow, all parts and percentages are given by weight, unless otherwise indicated.

### EXAMPLE 1

In this example, various results of biological tests have been illustrated which evidence the PPAR receptor transactivation properties of the compounds of the invention.

The comparative examples correspond to compounds which are described in EP-220,118 (comparative examples 1 and 2), or U.S. Pat. No. 5,023,363 (comparative examples 3 to 7), but which do not correspond to the conditions for the compounds of formula (I).

The biological tests carried out correspond to those described above. The method used to determine the AC₅₀ values was that described in Kliewer et al., Nature, 358, 771-774 (1992). Thus, the activating power via PPAR-α, PPAR-γ or PPAR-δ of molecules may be evaluated with a transactivation test in which HeLa cells were cotransfected with an expression vector encoding these receptors and a reporter plasmid containing a PPRE response element cloned upstream of part of an SV40 virus promoter and of the luciferase gene. The cotransfected cells were treated for 24 hours with the molecules to be tested and the luciferase activity was determined by luminescence.

Reference 1, reference molecule for PPAR-α was [4-chloro-6-(2,3-dimethylphenylamino)pyrimidin-2-ylsulphanyl]acetic acid;

Reference 2, reference molecule for PPAR-δ and PPAR-γ was 5-{4[2-(methylpyridin-2-ylamino)ethoxy]benzyl}thiazolidine-2,4-dione;

Comparative example 1 was 6-[butoxy(5,5,8,8-tetramethyl-5,6,7,8-tetrahydronaphthalen-2-yl)methyl]naphthalene -2-carboxylic acid;

Comparative example 2 was C-(6-carboxynaphthalen-2-yl)-C-(5,5,8,8-tetramethyl-5,6,7,8-tetrahydronaphthalen-2-yl)methylammonium;

Comparative example 3 was 6-(2,4-diisopropylbenzoyl)napthalene-2-carboxylic acid;

Comparative example 4 was 6-(6-methoxybiphenyl-3-carbonyl)napthalene-2-carboxylic acid;

Comparative example 5 was 6-(4-adamantan-1-yl-3-methoxybenzoyl)naphthalene-2-carboxylic acid;

Comparative example 6 was 4-[(3-adamantan-1-yl-4-methoxyphenyl)hydroxymethyl]benzoic acid;

Comparative example 7 was 4-(3-adamantan-1-yl-4-methoxybenzoyl)benzoic acid.

The results obtained in the PPAR-type receptor transactivation tests are grouped together in the following table:

TABLE

| Compounds | α | γ | β |
|---|---|---|---|
| Reference 1 | 100*(1.4)** | n.a. | n.a. |
| Reference 2 | n.a. | 100(0.07) | 100(0.13) |
| Compound 1 | 10 | 10 | 64 |
| Compound 2 | 48 (0.9) | 190(1.5) | 280(0.7) |
| Compound 3 | 63(1) | 148(0.36) | 277(0.3) |
| Compound 4 | 40(2) | 174(0.8) | 291(0.5) |
| Compound 5 | 16 | 36 | 92 |
| Compound 6 | 7 | 20 | 54 |
| Comparative Example | 0 | 7 | 28 |
| 1 | | | |
| Comparative Example | 1 | 13 | 1 |
| 2 | | | |
| Comparative Example | 6 | 3 | 19 |
| 3 | | | |
| Comparative Example | 6 | 2 | 23 |
| 4 | | | |
| Comparative Example | 6 | 3 | 8 |
| 5 | | | |
| Comparative Example | 4 | 7 | 16 |
| 6 | | | |
| Comparative Example | 0 | 0 | 23 |
| 7 | | | |

n.a. connotes “not active”
* % activation
()** AC₅₀ in μM

These results evidence the activation for the compounds of the invention for the various subtypes of PPAR-type receptors: PPAR-α, PPAR-β and PPAR-γ.

### EXAMPLE 2

In this example, various specific compositions according to the invention were formulated:

A—Oral Administration

(a) 0.2 g tablet:

| | |
|---|---|
| Compound 1 | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

(b) Oral suspension in 5 ml vials:

| | | | |
|---|---|---|---|
| Compound 5 | | 0.001 | g |
| Glycerine | | 0.500 | g |
| Sorbitol at 70% | | 0.500 | g |
| Sodium saccharinate | | 0.010 | g |
| Methyl para-hydroxybenzoate | | 0.040 | g |
| Flavoring | qs | | |
| Purified water | qs | 5 | m |

(c) 0.8 g tablet:

| | |
|---|---|
| Compound 2 | 0.500 g |
| Pregelatinized starch | 0.100 g |
| Microcrystalline cellulose | 0.115 g |
| Lactose | 0.075 g |
| Magnesium stearate | 0.010 g |

(d) Oral suspension in 10 ml vials:

| | | | |
|---|---|---|---|
| Compound 4 | | 0.200 | g |
| Glycerine | | 1.000 | g |
| Sorbitol at 70% | | 1.000 | g |
| Sodium saccharinate | | 0.010 | g |
| Methyl parahydroxybenzoate | | 0.080 | g |
| Flavoring | qs | | |
| Purified water | qs | 10 | ml |

B—Topical Administration

(a) Ointment:

| | |
|---|---|
| Compound 6 | 0.020 g |
| Isopropyl myristate | 81.700 g |
| Fluid liquid paraffin | 9.100 g |
| Silica (“Aerosil 200” marketed by | 9.180 g |
| DEGUSSA) | |

(b) Ointment:

| | | |
|---|---|---|
| Compound 2 | | 0.300 g |
| Petroleum jelly codex | qs | 100 g |

(c) Nonionic water-in-oil cream:

| | | |
|---|---|---|
| Compound 1 | | 0.100 g |
| Mixture of emulsive lanolin alcohols, | | 39.900 g |
| waxes and oils (“Anhydrous eucerin” | | |
| marketed by BDF) | | |
| Methyl para-hydroxybenzoate | | 0.075 g |
| Propyl para-hydroxybenzoate | | 0.075 g |
| Sterile demineralized water | qs | 100 g |

(d) Lotion:

| | |
|---|---|
| Compound 3 | 0.100 g |
| Polyethylene glycol (PEG 400) | 69.900 g |
| Ethanol at 95% | 30.000 g |

(e) Hydrophobic ointment:

| | | |
|---|---|---|
| Compound 5 | | 0.300 g |
| Isopropyl myristate | | 36.400 g |
| Silicone oil (“Rhodorsil 47 V 300” | | 36.400 g |
| marketed by RHONE-POULENC) | | |
| Beeswax | | 13.600 g |
| Silicone oil (“Abil 300,000 cst” | qs | 100 g |
| marketed by GOLDSCHMIDT) | | |

(f) Nonionic oil-in-water cream:

| | | |
|---|---|---|
| Compound 2 | 1.000 | g |
| Cetyl alcohol | 4.000 | g |
| Glyceryl monostearate | 2.500 | g |
| PEG 50 stearate | 2.500 | g |
| Shea butter | 9.200 | g |
| Propylene glycol | 2.000 | g |
| Methyl para-hydroxybenzoate | 0.075 | g |
| Propyl para-hydroxybenzoate | 0.075 | g |
| Sterile demineralized water qs | 100 | g |

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for treating the skin disorders/afflictions of an individual in need of such treatment, comprising administering to such individual, for such period of time as required to elicit the desired response, a thus-effective amount of at least one polycyclic aromatic compound having the structural formula (I): 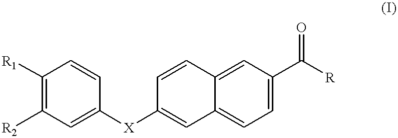
in which R is a hydrogen atom or an —OR₃ radical, wherein R₃ is as defined below; X is a ═C═O radical or a ═C═N—OH radical; R₁ and R₂, which may be identical or different, are each a hydrogen atom, a linear or branched alkyl radical having from 1 to 12 carbon atoms, an —OR₄ radical, an —S(O)ₙR₄ radical, an —OCOR₄ radical, or an —NHCOR₄ radical, wherein n and R₄ are as defined below, with the proviso that R₁ and R₂ may together form with the adjacent aromatic ring a 5- or 6-membered saturated or unsaturated ring optionally substituted with methyl groups and/or optionally interrupted by an oxygen or sulfur atom, a sulfone radical or a sulfoxide radical, and with the further proviso that, when X is a ═C═O radical, then R₁ and R₂ do not form with the adjacent aromatic ring a 5-membered ring substituted with methyl groups; R₃ is a hydrogen atom, a linear or branched alkyl radical having from 1 to 20 carbon atoms, or a mono- or polyhydroxyalkyl radical; n is 0, 1 or 2; R₄ is a hydrogen atom, a lower alkyl radical, or a phenyl radical; or salt or chiral analog thereof.

2. A regime or regimen for treating rosacea, intrinsic or chronological aging, a skin pigmentation disorder, a seborrhoeic function disorder, a barrier function of the skin disorder, an epidermal lipid secretion disorder, a wound healing disorder, a corticosteroid-induced cutaneous atrophy or ulcer, an immune system disorder, a cardiovascular system condition, and/or a lipid metabolism disorder, comprising administering to an individual in need of such treatment, for such period of time as required to elicit the desired response, a thus-effective amount of at least one polycyclic aromatic compound having the structural formula (I): 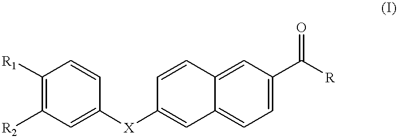
in which R is a hydrogen atom or an —OR₃ radical, wherein R₃ is as defined below; X is a ═C═O radical or a ═C═N—OH radical; R₁ and R₂, which may be identical or different, are each a hydrogen atom, a linear or branched alkyl radical having from 1 to 12 carbon atoms, an —OR₄ radical, an —S(O)ₙR₄ radical, an —OCOR₄ radical, or an —NHCOR₄ radical, wherein n and R₄ are as defined below, with the proviso that R₁ and R₂ may together form with the adjacent aromatic ring a 5- or 6-membered saturated or unsaturated ring optionally substituted with methyl groups and/or optionally interrupted by an oxygen or sulfur atom, a sulfone radical or a sulfoxide radical, and with the further proviso that, when X is a ═C═O radical, then R₁ and R₂ do not form with the adjacent aromatic ring a 5-membered ring substituted with methyl groups; R₃ is a hydrogen atom, a linear or branched alkyl radical having from 1 to 20 carbon atoms, or a mono- or polyhydroxyalkyl radical; n is 0 1 or 2; R₄ is a hydrogen atom , a lower alkyl radical, or a phenyl radical; or salt or chiral analog thereof.

3. The regime or regimen as defmed by claim 2, comprising treating hyperpigmentation, melasma, hypopigmentation, or vilitigo.

4. The regime or regimen as defined by claim 2, comprising treating a skin pigmentation disorder not related to solar radiation.

5. The regime or regimen as defined by claim 2, comprising treating hyperseborrhoea or seborrhoeic dermatitis.

6. The regime or regimen as defined by claim 2, comprising treating skin disorders in babies born before 33 weeks, lip fissures, chapped lips, or blisters.

7. The regime or regimen as defmed by claim 2, comprising treating keloids or hypertrophic scars.

8. The regime or regimen as defined by claim 2, comprising treating ulcers and erosions due to chemical or thermal burns, a bullous disorder, or a vascular or ischaemia disorder.

9. The regime or regimen as defined by claim 8, comprising treating venous, arterial, embolic or diabetic ulcers.

10. The regime or regimen as defined by claim 2, comprising treating type 1 diabetes mellitus, multiple sclerosis, lupus, a lupus-type disorder, glomerulonephritis, or AIDS.

11. The regime or regimen as defined by claim 2, comprising treating atherosclerosis or hypertension.

12. The regime or regimen as defined by claim 2, comprising treating obesity, hyperlipidaemia, or non-insulin-dependent diabetes.

13. The regime or regimen as defined by claim 1, said at least one polycyclic aromatic compound (I) comprising a PPAR receptor activator.

14. The regime or regimen as defined by claim 2, said at least one polycyclic aromatic compound (I) comprising a PPAR receptor activator.

15. The regime or regimen as defined by claim 2, said at least one polycyclic aromatic compound (I) comprising at least one lower alkyl radical selected from the group consisting of methyl, ethyl, isopropyl, butyl, tert-butyl and hexyl radicals.

16. The regime or regimen as defined by claim 2, said at least one polycyclic aromatic compound (I) comprising at least one linear or branched alkyl radical having from 1 to 20 carbon atoms selected from the group consisting of methyl, ethyl, propyl, 2-ethylhexyl, octyl, dodecyl, hexadecyl and octadecyl radicals.

17. The regime or regimen as defined by claim 2, said at least one polycyclic aromatic compound (I) comprising at least one monohydroxyalkyl radical selected from the group consisting of 2-hydroxyethyl, 2-hydroxypropyl and 3-hydroxypropyl radicals.

18. The regime or regimen as defined by claim 2, said at least one polycyclic aromatic compound (I) comprising at least one polyhydroxyalkyl radical selected from the group consisting of 2,3-dihydroxypropyl, 2,3,4-tri-hydroxybutyl and 2,3,4,5-tetrahydroxypentyl radicals and the pentaerythritol residue.

19. The regime or regimen as defined by claim 2, said at least one polycyclic aromatic compound (I) comprising Compound 1: 6-(5,6,7,8-tetrahydro-5,5,8,8-tetra-methylnaphthalene-2-carbonyl)naphthalene-2-carboxylic acid; Compound 2: 6-(2,2-dimethylchroman-6-carbonyl)naphthalene-2-carboxylic acid; Compound 3: 6-(4-tert-butylbenzoyl)naphthalene-2-carboxylic acid; Compound 4: 6-(5,6,7,8-tetrahydronaphthalene-2-carbonyl)naphthalene-2-carboxylic acid; Compound 5: 6-(4,4-dimethylchroman-6-carbonyl)naphthalene-2-carboxylic acid; or Compound 6: 6-[hydroxyimino(5,5,8,8-tetramethyl-5,6,7,8-tetrahydronaphthalen-2-yl)methyl]naphthalene-2-carboxylic acid.

20. The regime or regimen as defined by claim 2, said at least one polycyclic aromatic compound (I) comprising 6-(4-tert-butylbenzoyl)-naphthalene-2-carboxylic acid.

21. The regime or regimen as defined by claim 1, comprising administering said at least one polycyclic aromatic compound (I) via enteral or parenteral route.

22. The regime or regimen as defined by claim 1, comprising administering said at least one polycyclic aromatic compound (I) via topical or ocular route.

23. The regime or regimen as defined by claim 1, comprising coadministering to such individual an effective amount of at least one retinoid, vitamin D or derivative thereof, corticosteroid, agent for combating free radicals, α-hydroxy or α-keto acid or derivative thereof, or ion channel blocker.

24. Tablets, capsules, syrup, suspension, solution, powder, granules, emulsion, lipid or polymeric microspheres, nanospheres or vesicles comprising an amount effective for treating rosacea, intrinsic or chronological aging, a skin pigmentation disorder, a seborrhoeic function disorder, a barrier function of the skin disorder, an epidermal lipid secretion disorder, a wound healing disorder, a corticosteroid-induced cutaneous atrophy or ulcer, an immune system disorder, a cardiovascular system condition, and/or a lipid metabolism disorder, of at least one polycyclic aromatic compound having the structural formula (I): 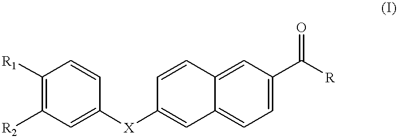
in which R is a hydrogen atom or an —OR₃ radical, wherein R₃ is as defined below; X is a ═C═O radical or a ═C═N—OH radical; R₁ and R₂, which may be identical or different, are each a hydrogen atom, a linear or branched alkyl radical having from 1 to 12 carbon atoms, an —OR₄ radical, an —S(O)ₙR₄ radical, an —OCOR₄ radical, or an —NHCOR₄ radical, wherein n and R₄ are as defined below, with the proviso that R₁ and R₂ may together form with the adjacent aromatic ring a 5- or 6-membered saturated or unsaturated ring optionally substituted with methyl groups and/or optionally interrupted by an oxygen or sulfur atom, a sulfone radical or a sulfoxide radical, and with the further proviso that, when X is a ═C═O radical, then R₁ and R₂ do not form with the adjacent aromatic ring a 5-membered ring substituted with methyl groups; R₃ is a hydrogen atom, a linear or branched alkyl radical having from 1 to 20 carbon atoms, or a mono- or polyhydroxyalkyl radical; n is 0, 1 or 2; R₄ is a hydrogen atom, a lower alkyl radical, or a phenyl radical, or salt or chiral analog thereof, formulated into an enterally/parenterally administrable, cosmetically/pharmaceutically acceptable vehicle, diluent or carrier therefor.

25. A salve, cream, emulsion, milk, ointment, powder, impregnated pad, solution, gel, spray, lotion, suspension, lipid or polymeric microspheres, nanospheres, vesicles, patch, hydrogel, soap or shampoo comprising an amount effective for treating rosacea, intrinsic or chronological aging, a skin pigmentation disorder, a seborrhoeic function disorder, a barrier function of the skin disorder, an epidermal lipid secretion disorder, a wound healing disorder, a corticosteroid-induced cutaneous atrophy or ulcer, an immune system disorder, a cardiovascular system condition, and/or a lipid metabolism disorder, of at least one polycyclic aromatic compound having the structural formula (I): 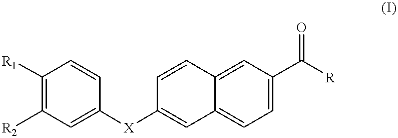
in which R is a hydrogen atom or an —OR₃ radical, wherein R₃ is as defined below; X is a ═C═O radical or a ═C═N—OH radical; R₁ and R₂, which may be identical or different, are each a hydrogen atom, a linear or branched alkyl radical having from 1 to 12 carbon atoms, an —OR₄ radical, an —S(O)ₙR₄ radical, an —OCOR₄ radical, or an —NHCOR₄ radical, wherein n and R₄ are as defined below, with the proviso that R₁ and R₂ may together form with the adjacent aromatic ring a 5- or 6-membered saturated or unsaturated ring optionally substituted with methyl groups and/or optionally interrupted by an oxygen or sulfur atom, a sulfone radical or a sulfoxide radical, and with the further proviso that, when X is a ═C═O radical, then R₁ and R₂ do not form with the adjacent aromatic ring a 5-membered ring substituted with methyl groups; R₃ is a hydrogen atom, a linear or branched alkyl radical having from 1 to 20 carbon atoms, or a mono- or polyhydroxyalkyl radical; n is 0, 1 or 2; R₄ is a hydrogen atom, a lower alkyl radical, or a phenyl radical, or salt or chiral analog thereof, formulated into a topically applicable, cosmetically/pharmaceutically acceptable vehicle, diluent or carrier therefor.

26. The formulation as defined by claim 24, further comprising an effective amount of at least one retinoid, vitamin D or derivative thereof, corticosteroid, agent for combating free radicals, α-hydroxy or α-keto acid or derivative thereof, or ion channel blocker.

27. The formulation as defined by claim 25, further comprising an effective amount of at least one retinoid, vitamin D or derivative thereof, corticosteroid, agent for combating free radicals, α-hydroxy or α-keto acid or derivative thereof, or ion channel blocker.
